(19) United States
(12) Patent Application Publication
(10) Pub. No.: US 2006/0068010 A1
(43) Pub. Date: Mar. 30, 2006

(54) Method for improving the bioavailability of orally delivered therapeutics

(76) Inventors: Stephen Turner, Snoqualmie, WA (US); Jyo Ravishankar, Bellevue, WA (US); Reza Fassihi, Fort Washington, PA (US)

Correspondence Address: RATNERPRESTIA, P O BOX 980, VALLEY FORGE, PA, 19482-0980, US

(21) Appl. No.: 11/238,775

(22) Filed: Sep. 29, 2005

Related U.S. Application Data
(60) Provisional application No. 60/614,893, filed on Sep. 30, 2004.
(60) Provisional application No. 60/625,277, filed on Nov. 5, 2004.
(60) Provisional application No. 60/637,250, filed on Dec. 17, 2004.

(51) Int. Cl. A61K 9/26

(52) U.S. Cl. 424/469

(57) ABSTRACT

The disclosed invention is a method and composition for improving the bioavailability of a pharmaceutically active ingredient comprising an oral dosage form consisting essentially of a granulation of active ingredient, amino acid, and hydrophilic polymer, wherein the granulation is dispersed in an immediate release or extended release excipient.

The present invention claims the benefit of U.S. Provisional Application Nos. 60/614,893, filed Sep. 30, 2004; 60/625,277, filed Nov. 5, 2004; and 60/635,250, filed Dec. 17, 2004.

### FIELD OF THE INVENTION

The present invention is directed to a method for improving the oral delivery of pharmaceutically active compounds having limited bioavailability due to limited solubility or limited permeability. More specifically the present invention is directed to improving the bioavailability and absorption of such compounds when administered orally.

### BACKGROUND OF THE INVENTION

Bioavailability is defined as the fraction of unchanged drug reaching the systemic circulation following administration by any route. (B. Katzung, Basic & Clinical Pharmacology, Norwalk Conn.: Appleton & Lange 1995, page 39). Poor drug bioavailability can result from low drug solubility, low drug permeability, or both, and any metabolism or degradation of the drug before it reaches the circulation. The dosage form of an active ingredient can have a great effect on its solubility and permeability, thereby affecting bioavailability. The Biopharmaceutics Classification System classifies drugs into four groups: Class 1: high permeability, high solubility; Class 2: high permeability, low solubility; Class 3: low permeability, high solubility; and Class 4: low permeability, low solubility. (H. van de Waterbeemd. in Oral Drug Absorption, Prediction and Assessment, J. Dressman and H. Lennernas, Eds., New York: Marcel Dekker, Inc., 2000, page 38.) Low bioavailability is often associated with oral dosage forms of Class 2-4 drugs, i.e., drugs with low solubility, low permeability, or both.

A pharmaceutically active compound is conventionally classified as highly soluble when the largest dose of the compound is soluble in less than 250 mL water over a pH range from 1.0 to 7.5. Soluble compounds have a solubility range of greater than or equal to 33 mg/mL. Sparingly soluble compounds have a range from 10-33 mg/mL, slightly soluble compounds from 1-10 mg/mL, and very slightly soluble compounds from 0.1-1 mg/mL (Kasim et al., Mol. Pharm. 1: 85-96, 2004). Compounds with solubilities below 1 mg/mL are classified as practically insoluble. Sparingly soluble or less than sparingly soluble compounds, hereinafter referred to as “low solubility” compounds, are frequently difficult to formulate into dosage forms that promote the bioavailability of the active ingredient.

The bioavailability of low solubility drugs may be related, in part, to drug particle size. Reducing particle size increases the surface area of the compound and can improve the dissolution properties of the drug to allow a wider range of formulation approaches and delivery technologies. Conventional methods of particle size reduction, such as comminution and spray drying, rely upon mechanical stress to disaggregate the active compound. The critical parameters of comminution are well-known to the industry, thus permitting an efficient, reproducible and economic means of particle size reduction. However, the mechanical forces inherent to comminution, such as milling and grinding, often impart significant amounts of physical stress upon the drug product which may induce degradation. The thermal stress which may occur during comminution and spray drying is also a concern when processing thermo-sensitive or unstable active compounds. Moreover, traditional comminution and micronizing techniques may not be able to reduce particle size sufficiently to significantly improve bioavailability or permeability. The present invention may utilize a micronized or other fine particle sized material, but demonstrates improvement in bioavailability independent of such particle size reduction techniques.

Fassihi and Durig, in U.S. Pat. Nos. 6,517,868 and 6,936,275, disclosed a means for providing extended release of low solubility compounds through granulating certain active ingredients with a polymer and an amino acid and dispersing the resulting granulation in a more rapidly hydrating polymer. However, the disclosures of these patents is directed to the use of an extended release dosage form that provides zero order release of low solubility compounds over an extended period of time. These patents require both a more rapidly hydrating extra-granular polymer and a more slowly hydrating intra-granular polymer to effect zero order release of the active ingredient. Neither of these patents discloses a means for improving the bioavailability of low solubility compounds in immediate release dosage forms or improving the permeability of low permeability active ingredients in immediate or extended release dosage forms.

In order to reach its site of action in the body, a drug must first be absorbed into the blood from its site of administration. Orally administered drugs are generally absorbed into the blood from the gastrointestinal (GI) tract and must pass through the cell membranes of GI tract cells and blood vessel cells to enter the blood stream. The inherent ability of a compound to pass through a barrier such as a cell membrane, is known as permeability. Highly permeable compounds are classified as those compounds that demonstrate greater than 90% absorption of the administered dose. Low permeability compounds demonstrate less than 20% absorption of the administered dose. While the present invention, in one aspect is directed to low permeability compounds in particular, one skilled in the art will appreciate that the present invention may also be of benefit to those compounds whose permeability is greater than that of low permeability compounds, such as ondansetron.

Cell membranes are made up of a lipid bilayer, and the physicochemical properties of a drug compound determines how easily the compound can permeate the cell membranes and be absorbed from the GI tract into the circulation. Lipid-soluble compounds (hydrophobic compounds) will easily permeate the lipid bilayer, but compounds soluble in aqueous solutions (hydrophilic compounds) will not. Hydrophobicity is determined by the electrical charges of a chemical compound. Highly charged compounds (polar compounds) tend to be hydrophilic and uncharged compounds (nonpolar compounds) tend to be hydrophobic. One way to increase the hydrophobicity of a drug, and thus its ability to be absorbed into the bloodstream, is to reduce its electrical charges. It has been found that this phenomenon is facilitated by granulating a low solubility or low permeability compound with an amino acid and an intra-granular hydrophilic polymer then dispersing the granulation in an immediate release excipient such as microcrystalline cellulose, or in a hydrophilic sustained release polymer having a viscosity greater than the viscosity of the intra-granular polymer.

### SUMMARY OF THE INVENTION

The invention provides a method and composition for improving bioavailability of a pharmaceutically active ingredient comprising orally administering to a subject in need of said active ingredient a dosage form consisting essentially of
a) a granulation comprising granules of a low solubility or low permeability active ingredient, at least one amino acid, and at least one intra-granular hydrophilic polymer;
b) one or more formulation excipients in which a therapeutic amount of said granulation is substantially uniformly dispersed, said excipient comprising:
   (i) an immediate release excipient selected from the group consisting of microcrystalline cellulose, sodium carboxymethyl cellulose, sodium starch glycolate, corn starch and combinations of such excipients when said dosage form is an immediate release dosage form, or
   (ii) a sustained release excipient comprising a polymer having a viscosity higher than the viscosity of said intra-granular polymer;
c) said composition being in the form of a capsule or compressed tablet.

### DETAILED DESCRIPTION OF THE INVENTION

The present invention provides a method and composition for improving the bioavailability or absorption of an orally administered pharmaceutically active ingredient that is a low solubility or low permeability compound, or becomes a low solubility or low permeability compound under conditions found at the situs of absorption. For purposes of the invention, a low solubility compound is one which is sparingly or less than sparingly soluble in water. The active ingredient is granulated with at least one amino acid and with a hydrophilic polymer and the resulting granulation is blended with various formulation excipients. The product may then be filled into capsules, or compressed into tablets to provide an oral dosage form containing a therapeutic amount of the active ingredient. The dosage form may be an immediate release or extended release dosage form. In an immediate release dosage form, release of the active ingredient proceeds promptly after the dosage form is administered. In an extended release dosage form, the components of the formulation are selected to extend release of the active ingredient after administration of the dosage form.

Low solubility compounds include BCS Class 2 and BCS Class 4 compounds. Class 2 compounds include, for example, amiodarone HCl, atazanavir sulfate, atorvastatin, azithromycin, benazepril HCl, bicalutamide, candesartan cilexetil, carbamazepine, carisoprodol, carvedilol, celecoxib, clarithromycin, diazepam, divalproex sodium, docetaxel, donepezil HCL, efavirenz, etodolac, ezetimibe, fenofibrate, finasteride, gemfibrozil, glimepiride, glyburide, ibuprofen, indapamide, indomethacin, irbesartan, ketoconazole, lansoprazole, loratadine, lovastatin, meclizine HCL, metaxalone, moxifloxacin HCl, mycophenolate mofetil, nabumetone, nelfinavir mesylate, olmesartan medoxomil, pioglitazone HCl, prednisone, raloxifene HCl, risperidone, ritonavir, rofecoxib, simvastatin, spironolactone, tacrolimus, temazepam, valdecoxib, valsartan, ziprasidone HCl.

BCS Class 4 compounds include, for example, acyclovir, allopurinol, aspirin, cefdinir, cefprozil, cephalexin, clindamycin HCl, doxycycline hyclate, famotidine, felodipine, furosemide, glipizide, linezolid, meloxicam, mesalamine, methocarbamol, methotrexate, nifedipine, nitrofurantoin, olanzapine, oxcarbazepine, phenobarbital, sildenafil citrate, tadalafil, temozolomide, tetracycline, theophylline.

Low permeability compounds include Class 3 and Class 4 compounds. Class 3 compounds include, for example, albuterol, alendronate sodium, amlodipine besylate, amoxicillin, atenolol, baclofen, buspirone HCl, captopril, carboplatin, ceftriaxone, ciprofloxacin, ciprofloxacin hcl, colchicine, fluconazole, folic acid, gabapentin, gemcitabine HCl, granisetron HCl, hydrochlorothiazide, hyoscyamine sulfate, lamivudine, lamotrigine, levetiracetam, levofloxacin, lisinopril, metformin HCl, metronidazole, minocycline HCl, morphine sulfate, niacin, oxaliplatin, oxycodone HCl, oxycontin, penicillin VK, progesterone, ranitidine, risedronate sodium, rosiglitazone, sumatriptan, terazosin HCl, thalidomide, timolol maleate, topiramate, valacyclovir HCl, zoledronic acid, zolpidem.

In the examples below the invention is illustrated with respect to raloxifene, atenolol, ondansetron, and rosiglitozone.

Bioavailability may be determined by administering a dosage form to a human or animal subject and measuring the concentration of unchanged active ingredient in the bloodstream over time. Both the rate and extent of drug absorption determine the shape of the curve of a concentration vs. time plot. The area under the curve (AUC) is directly proportional to the total amount of unchanged drug in the systemic circulation and is the most reliable measure of bioavailability. The time at which maximum systemic unchanged drug concentration occurs (Tmax) can be used as an indication of absorption rate, i.e., a slower absorption rate will result in a later peak time. The maximum systemic unchanged drug concentration is referred to as Cmax.

While not being bound by any particular theory of operation, it is believed that the criteria for choosing an amino acid for the granulation relate to the permeability characteristics, for example, polarity, of the active ingredient and the polarity of the amino acid side chains. Permeability characteristics of a drug can be quantified based on the partition of the drug in a water-n-octanol mixture. Log P, the partition coefficient of the drug compound, is the log of the equilibrium concentration of the drug in the n-octanol and water layers. Log P increases proportionally to the hydrophilicity of a compound. A compound with log P greater than or equal to 1.72 is a high permeability compound. A compound with log P less than 1.72 is a low permeability compound. (N. Kasim et al., Molecular Pharmaceutics, 1: 85-96, 2004; Pliska et al., J. Chromatography 216: 79-92, 1981).

Amino acids, like drug compounds, contain electrically charged chemical groups and are classified based on the extent of their polarity. Amino acids with nonpolar (uncharged, hydrophobic) side groups are valine, leucine, isoleucine, methionine, and phenylalanine. Amino acids with polar (charged, hydrophilic) side groups are asparagine, glutamine, histidine, lysine, arginine, aspartic acid and glutamic acid. Glycine has no side groups and is considered a neutral amino acid. Alanine, serine, threonine, tyrosine, tryptophan, cysteine, and proline are intermediate between the polar and nonpolar amino acids.

The selected amino acid should form a non-covalent complex with the active ingredient and reduce its charge, thereby increasing membrane permeability and absorption for the drug-amino acid complex. For example, if the active ingredient is a highly-charged polar compound, an amino acid would be chosen that would mask the charge groups of the active ingredient, thereby rendering the resultant drug-amino acid complex more permeable to epithelial cell membranes and increasing absorption. The selected amino acid(s) may be α-amino acids, β-amino acids, or combinations of α-amino acids and β-amino acids.

While some routine experimentation may be necessary with respect to selection of the optimal amino acid for a given compound, reference may be made to published tables setting forth relative values of hydrophobicity/hydrophilicity for amino acids, such as that set forth in Table 1. In this table, the values are normalized to glycine so that the most hydrophobic amino acid has a value of 100 relative to glycine, which is neutral and has a value of zero. Such tables and the hydropathy characteristics described therein are well known and understood by those skilled in the art, as are methods for determining drug solubility and absorption. (Pliska et al., 3. Chromatography 216: 79-92, 1981; N. Kasim et al., Molecular Pharmaceutics, 1: 85-96, 2004; H. van de Waterbeemd, in Oral Drug Absorption, Prediction and Assessment, J. Dressman and H. Lennernas, Eds., New York: Marcel Dekker, Inc., 2000, pages 31-49). (Table 1 is derived from http://www.sigmaaldrich.com).

TABLE 1
Hydrophobicity Index for Amino Acids

| | |
|---|---|
| At pH 2 | At pH 7 |

Very Hydrophobic

| | | | |
|---|---|---|---|
| Leu | 100 | Phe | 100 |
| Ile | 100 | Ile | 99 |
| Phe | 92 | Trp | 97 |
| Trp | 84 | Leu | 97 |
| Val | 79 | Val | 76 |
| Met | 74 | Met | 74 |

Hydrophobic

| | | | |
|---|---|---|---|
| Cys | 52 | Tyr | 63 |
| Tyr | 49 | Cys | 49 |
| Ala | 47 | Ala | 41 |

Neutral

| | | | |
|---|---|---|---|
| Thr | 13 | Thr | 13 |
| Glu | 8 | His | 8 |
| Gly | 0 | Gly | 0 |
| Ser | −7 | Ser | −5 |
| Gln | −18 | Gln | −10 |
| Asp | −18 | | |

Hydrophilic

| | | | |
|---|---|---|---|
| Arg | −26 | Arg | −14 |
| Lys | −37 | Lys | −23 |
| Asn | −41 | Asn | −28 |
| His | −42 | Glu | −31 |
| Pro | −46 | Pro | −46* |
| | | Asp | −55 |

*based on pH 2.0

The weight ratio of amino acid to active ingredient in the granulation may be from about 1:1 to about 10:1, and generally from about 2:1 to about 4:1.

The granulation also includes at least one intra-granular hydrophilic polymer, such as hydroxypropyl methylcellulose (HPMC) or guar gum, in a weight ratio from about 1:1 to about 10:1, or from about 1:1 to 1:3 polymer to active ingredient. Low viscosity hydrophilic polymers, for example, polymers having a viscosity in the range of about 100 to about 5000 cps, such as HPMC K100LV and E4 MP, are employed as an intra-granular polymer.

Granulation of pharmaceutically active ingredients with conventional pharmaceutical hydrophilic polymers, such as HPMC, and polysaccharides, such as guar gum, is well known. Means of granulating both hydrophilic and hydrophobic pharmaceutically active compounds are also well known in the art, and may be used to prepare the granulations of this invention containing active ingredient(s), amino acid(s), and polymer(s).

Conventional extra-granular excipients such as microcrystalline cellulose (MCC), di-calcium phosphate (DCP), sodium carboxymethyl cellulose, sodium starch glycolate, and corn starch may be used in the immediate release formulation. In extended release formulations, the excipient comprises a polymer having a viscosity that is substantially greater than the viscosity of the intra-granular polymer, in particular, polymers having a viscosity ranging from about 5000 cps to about 100,000 cps, such as HPMC K15M and HPMC K100MP.

Preferably, the weight ratio of granulation to matrix excipients ranges from about 1:10 to about 1:50. The excipients may also include a lubricant, such as magnesium stearate. A therapeutic amount of the active ingredient is uniformly dispersed in the excipients. The final blended product may be placed in capsules or compressed and tabletted by conventional methods.

Following oral ingestion of the dosage form by a subject, the amount of the active ingredient absorbed from the dosage form is greater than the amount of active ingredient absorbed by the subject from a corresponding “control” dosage form having the same active ingredient and excipients and having no amino acid. Absorption is determined by the AUC for a selected interval of a concentration versus time plot.

### EXAMPLE 1

In one embodiment of an immediate release formulation, the polymer, amino acid and active pharmaceutical ingredient are present in the granulation in a weight ratio of 1:2:1 prior to being blended with microcrystalline cellulose (MCC), as an excipient, and silica, as a flow agent, as shown in Table 2.

TABLE 2

| | |
|---|---|
| Granulation | Blend |

| | | | |
|---|---|---|---|
| Active ingredient | 150 g | Granulation | 60 mg |
| Glycine | 300 g | MCC | 240 mg |
| HPMC K100LV | 150 g | Silica | 6 mg |
| Total | 600 g | | *306 mg |

*Total per tablet

### EXAMPLE 2

An example of a low solubility compound capable of improved bioavailability in an immediate release formulation, such as Example 1, is raloxifene hydrochloride. Raloxifene, (6-hydroxy-2-(4-hydroxyphenyl)-3-[4-(2-piperidinoethoxy) benzoyl]benzo[p]-thiophene), is a second generation selective estrogen receptor modulator. Raloxifene has been shown to be useful in the treatment of osteoporosis and may be useful in other estrogen-related pharmacology. In its hydrochloride-salt form, raloxifene is classified as a “very slightly soluble,” (at approximately 0.3 mg/mL) compound.

Tablets containing 3 mg active ingredient were manufactured according to the formulation in Example 1 using a manually-advanced rotary press. Tablets and control pellets were administered via oral gavage to 6 rat subjects, each weighing 350-375 g. Plasma samples were captured via jugular cannula pre-dose and at 5, 10, 15, 30, 45, 60, 90 and 120 min post-dose. Plasma levels of raloxifene were measured using LC-MS/MS optimized for specificity and sensitivity and pharmacokinetic parameters were determined using WinNonlin software.

Plasma levels were evaluated for raloxifene HCl versus unmodified control formulations (3 mg raloxifene and MCC), as shown in Table 3.

TABLE 3

| | AUC | | Cmax | | Tmax | |
|---|---|---|---|---|---|---|
| | (min * ng/mL) | | (ng/mL) | | (min) | |
| | Mean | StdDev | Mean | StdDev | Mean | StdDev |
| Raloxifene | 933.2 | 706.8 | 15 | 8.1 | 93 | 45.5 |
| (no amino acid) | | | | | | |
| Raloxifene | 1418.2 | 1172.4 | 28.3 | 22.8 | 120 | 0 |
| (Glycine) | | | | | | |

Although both groups showed variability among the animals, the mean AUC for the formulation of the present invention was almost 50% higher than the mean AUC for the control group. These results demonstrate an improvement in the bioavailability of a low solubility compound, raloxifene, as evidenced by an increase in AUC when compared with the control formulation.

### EXAMPLE 3

An example of a low permeability compound that is capable of improved absorption in an immediate release formulation, such as Example 1, is atenolol hydrochloride. Atenolol, (benzeneacetamide, 4-[2′-hydroxy-3′-[(1-methylethyl) amino]propoxy]-), is a synthetic, beta₁-selective (cardioselective) adrenoreceptor blocking agent. Atenolol has been shown to be useful in the management of hypertension. In humans, absorption of an oral dose is rapid, but incomplete. Only about 50% of an oral dose is absorbed from the gastrointestinal tract, and the remainder is excreted.

Tablets containing 3 mg active drug were manufactured according to the formulation in Example 1 using a manually-advanced rotary press. Glycine was selected as the amino acid for formulation 1 and phenylalanine was selected as the amino acid for formulation 2. Tablets and control pellets were administered via oral gavage to 6 rat subjects, each weighing 350-375 g. Plasma samples were captured via jugular cannula pre-dose and at 5, 10, 15, 30, 45, 60, 90 and 120 minutes following administration of each dose. Plasma levels of atenolol were measured using LC-MS/MS optimized for specificity and sensitivity and pharmacokinetic parameters were determined using WinNonlin software.

Plasma levels were evaluated for Atenolol versus control formulations, as shown in Table 4.

TABLE 4

| | AUC | | Cmax | | | |
|---|---|---|---|---|---|---|
| | (min * ng/mL) | | (ng/mL) | | Tmax (min) | |
| | Mean | SD | Mean | SD | Mean | SD |
| Atenolol | 10449.2 | 2343.2 | 153.2 | 30.8 | 90.0 | 37.9 |
| (no amino | | | | | | |
| acid) | | | | | | |
| Atenolol | 12516.6 | 7599.1 | 201.6 | 97.3 | 102.5 | 30.6 |
| (Glycine) | | | | | | |
| Atenolol | 14176.9 | 11564.5 | 170.2 | 131.9 | 95.0 | 22.6 |
| (Phenylalanine) | | | | | | |

AUC: Area under the curve; Cmax: Estimated maximum plasma concentration; Tmax: Time of maximum observed concentration; SD: Standard deviation.

Although variability among animals was high, permeability was increased in animals administered the granulation formulation of the present invention as evidenced by the improvement in AUC.

### EXAMPLE 4

Another example of a low permeability compound whose bioavailability and absorption could be improved by the disclosed immediate release dosage form is ondansetron. Ondansetron, (+/−) 1,2,3,9-tetrahydro-9-methyl-3-[2-methyl-1H-imidazol-1-yl]-4H-carbazol-4-one, monohydrochloride, dehydrate, is a selective %-HT3 antagonist. Ondansetron has been shown to be useful in the treatment of emesis resulting from cyclophosphamide-based chemotherapy and may be useful in other nausea prevention. In its base form, ondansetron is also a low solubility compound at pH greater than 5.0.

An immediate release formulation for ondansetron and bioavailability data for the formulation is shown in Table 5.

TABLE 5

| | |
|---|---|
| Granulation | Blend |

| | | | |
|---|---|---|---|
| Ondansetron | 150 g | Granulation | 40 mg |
| Aspartic acid | 300 g | MCC | 100 mg |
| HPMC K100LV | 300 g | Silica | 3 mg |
| Total | 750 g | | *143 mg |

| Bioavailability | Cmax | AUC |
|---|---|---|
| Formulation | 933 ng/mL | 1418 ng/mL |
| Control | 15 ng/mL | 28 ng/mL |

*Total per tablet.

### EXAMPLE 5

Rosiglitazone maleate is an example of a low permeability and low solubility drug that is suitable for the immediate release formulation described in Example 1. Rosiglitazone, (±)-5-[[4-[2-(methyl-2-pyridinylamino)ethoxy]phenyl]methyl]-2,4-thiazolidinedione, (Z)-2-butenedioate (1:1), improves sensitivity to insulin in muscle and adipose tissue and inhibits hepatic gluconeogenesis. Solubility of Rosiglitazone maleate decreases with increased pH in the physiological range. Improved immediate release formulations of rosiglitazone can be prepared as described in Table 6.

TABLE 6

| | |
|---|---|
| Granulation | Blend |

| | | | |
|---|---|---|---|
| Rosiglitazone | 150 g | Granulation | 60 mg |
| maleate | | | |
| Glycine | 300 g | MCC | 240 mg |
| HPMC K100LV | 150 g | Silica | 6 mg |
| Total | 600 g | | *306 mg |
| Rosiglitazone | 150 g | Granulation | 60 mg |
| maleate | | | |
| Aspartic acid | 300 g | MCC | 240 mg |
| HPMC K100LV | 150 g | Mg stearate | 6 mg |
| Total | 600 g | | *306 mg |

*Total per tablet

### EXAMPLE 6

An extended release formulation for low permeability active ingredients can be prepared by including a high viscosity more slowly hydrating hydrophilic polymer in addition to the low viscosity more rapidly hydrating hydrophilic polymer.

In this embodiment, a low viscosity hydrophilic polymer, amino acid and active ingredient would be present in the granulation, which would then be blended with a high viscosity polymer and magnesium stearate, as a lubricant. This formulation is set forth in Table 7.

TABLE 7

| | |
|---|---|
| Granulation | Blend |

| | | | |
|---|---|---|---|
| Active | 100 g | Granulation | 240 mg |
| Glycine | 400 g | HPMC K15M | 60 mg |
| HPMC K100Lv | 100 g | Mg stearate | 3 mg |
| Total | 600 g | | *303 mg |

*Total per tablet

### EXAMPLE 7

Low permeability active ingredients suitable for the extended release formulation of Example 5 include ondansetron and rosiglitazone maleate. These extended release formulations could be prepared as set forth in Table 8.

TABLE 8

| | |
|---|---|
| Granulation | Blend |

| | | | |
|---|---|---|---|
| Ondansetron | 100 g | Granulation | 120 mg |
| Aspartic acid | 200 g | HPMC K15M | 240 mg |
| HPMC K100LV | 200 g | Mg stearate | 3 mg |
| Total | 500 g | | *363 mg |
| Rosiglitazone maleate | 100 g | Granulation | 240 mg |
| Glycine or aspartic acid | 400 g | HPMC K15M | 60 mg |
| HPMC K100LV | 100 g | Mg stearate | 3 mg |
| Total | 600 g | | *303 mg |

*Total per tablet

While selected embodiments of the invention have been shown and described herein, it will be understood that such embodiments are provided by way of example only. Numerous variations, changes and substitutions will occur to those skilled in the art without departing from the spirit of the invention. Accordingly, it is intended that the claims will cover all such variations as fall within the spirit of the invention.

### EXAMPLE 8

A dosage form of raloxifene HCl formulated according to Example 1 was administered to 19 post-menopausal human female subjects. The in vivo behavior of immediate release dosages of the formulation were compared to the in vivo behavior of an immediate release formulation comprising identical excipients but containing no amino acids. Following an overnight fast of at least 10 hours, each subject received one 45 mg dose of one of the above described immediate release tablets administered with 240 mL of ambient temperature water. Twenty-one blood samples were taken at specific intervals up to 72 hours after dosing.

Plasma levels of raloxifene were measured using LC-MS/MS optimized for specificity and sensitivity and pharmacokinetic parameters were determined using SAS software.

Plasma levels were evaluated for raloxifene HCl in the test formulation versus unmodified control formulations (identical excipients containing no amino acids), as shown in Table 9.

TABLE 9

| | AUC, 0-inf | | | | | |
|---|---|---|---|---|---|---|
| | (min * pg/mL) | | Cmax (ng/mL) | | Tmax (h) | |
| | Mean | StdDev | Mean | StdDev | Mean | Min-max |
| Raloxifene | 12376.02 | 7273.63 | 414.14 | 331.99 | 7 | 5-36 |
| (no amino | | | | | | |
| acid) | | | | | | |
| Raloxifene | 15783.69 | 8954.04 | 342.37 | 185.78 | 10 | 5-24 |
| (Glycine/ | | | | | | |
| Isoleucine) | | | | | | |

What is claimed is:

1. A method for improving bioavailability of a pharmaceutically active ingredient comprising orally administering to a subject in need of said active ingredient a dosage form consisting essentially of
a. a granulation comprising granules of a low solubility or low permeability active ingredient, at least one amino acid, and at least one intra-granular hydrophilic polymer;
b. one or more formulation excipients in which a therapeutic amount of said granulation is substantially uniformly dispersed, said excipient comprising:
i. an immediate release excipient selected from the group consisting of microcrystalline cellulose, sodium carboxymethyl cellulose, sodium starch glycolate, corn starch and combinations of such excipients when said dosage form is an immediate release dosage form, or
ii. a sustained release excipient comprising a polymer having a viscosity higher than the viscosity of the said intra-granular polymer;
c. said composition being in the form of a capsule or compressed tablet.

2. The method of claim 1, wherein the amount of the active ingredient absorbed from the dosage form is greater than the amount of active ingredient absorbed by the subject from a corresponding dosage form having the same active ingredient and excipients and having no amino acid.

3. The method of claim 2, wherein the AUC of said dosage form is increased over that for said dosage form having no amino acid in the granulation.

4. The method of claim 2, wherein said immediate release excipient comprises microcrystalline cellulose.

5. The method of claim 2, wherein said sustained release excipient comprises HPMC.

6. The method of claim 1, wherein said active ingredient comprises a low solubility active ingredient.

7. The method of claim 1, wherein said active ingredient comprises a low permeability active ingredient.

8. The method of claim 1, wherein said active ingredient is raloxifene, ondansetron, atenolol or rosiglitazone.

9. The method of claim 1, wherein said active ingredient is raloxifene.

10. The method of clam 1, wherein said active ingredient is atenolol.

11. The method of claim 1, wherein said active ingredient is ondansetron.

12. The method of claim 1, wherein said active ingredient is rosiglitazone.

13. The method of claim 1, wherein the ratio of the amino acid to the active ingredient is from about 1:1 to about 10:1.

14. The method of claim 13, wherein the ratio of the amino acid to the active ingredient is from about 2:1 to about 4:1.

15. The method of claim 13, wherein said amino acid is selected from the group consisting of aspartate, glutamate, lysine, arginine, asparagine, glutamine, histidine, serine, threonine, glycine, alanine, tyrosine, cysteine, proline, methionine, valine, tryptophan, phenylalanine, leucine, and isoleucine.

16. The method of claim 15, wherein said amino acid is selected from the group consisting of glycine, aspartate, and phenylalanine.

17. The method of claim 1, wherein the weight ratio of the intra-granular polymer to the active ingredient is from about 1:1 to about 10:1.

18. The method of claim 17, wherein the weight ratio of the intra-granular polymer to the active ingredient is from about 1:1 to 1:3.

19. The method of claim 1, wherein the hydrophilic intra-granular polymer has a viscosity in the range of about 100 to about 5000 cps.

20. The method of claim 19, wherein the intra-granular polymer comprises HPMC K100LV.

21. A composition comprising an oral solid dosage form which provides improved bioavailability for an orally administered low solubility or low permeability pharmaceutically active ingredient, said dosage form consisting essentially of
d. a granulation comprising granules of a low solubility or low permeability active ingredient, at least one amino acid, and at least one intra-granular hydrophilic polymer;
e. one or more formulation excipients in which a therapeutic amount of said granulation is substantially uniformly dispersed, said excipient comprising
i. an immediate release excipient selected from the group consisting of microcrystalline cellulose, sodium carboxymethyl cellulose, sodium starch glycolate, corn starch and combinations of such excipients when said dosage form is an immediate release dosage form, or
ii. a sustained release excipient comprising a polymer having a viscosity higher than the viscosity of the said intra-granular polymer;
f. said composition being in the form of a capsule or a compressed tablet.

22. The composition of claim 21, wherein the amount of the active ingredient absorbed from the dosage form is greater than the amount of active ingredient absorbed by the subject from a corresponding dosage form having the same active ingredient and excipients and having no amino acid.

23. The composition of claim 22, wherein the AUC of said dosage form is increased over that for said dosage form having no amino acid in the granulation.

24. The composition of claim 22, wherein said immediate release excipient comprises microcrystalline cellulose.

25. The composition of claim 22, wherein said sustained release excipient comprises HPMC.

26. The composition of claim 21, wherein said active ingredient comprises a low solubility active ingredient.

27. The composition of claim 21, wherein said active ingredient comprises a low permeability active ingredient.

28. The composition of claim 21, wherein said active ingredient is raloxifene, ondansetron, atenolol or rosiglitazone.

29. The composition of claim 21, wherein said active ingredient is raloxifene.

30. The composition of claim 21, wherein said active ingredient is atenolol.

31. The composition of claim 21, wherein said active ingredient is ondansetron.

32. The composition of claim 21, wherein said active ingredient is rosiglitazone.

33. The composition of claim 21, wherein the ratio of the amino acid to the active ingredient is from about 1:1 to about 10:1.

34. The composition of claim 33, wherein the ratio of the amino acid to the active ingredient is from about 2:1 to about 4:1.

35. The composition of claim 33, wherein said amino acid is selected from the group consisting of aspartate, glutamate, lysine, arginine, asparagine, glutamine, histidine, serine, threonine, glycine, alanine, tyrosine, cysteine, proline, methionine, valine, tryptophan, phenylalanine, leucine, and isoleucine.

36. The composition of claim 35, wherein said amino acid is selected from the group consisting of glycine, aspartate, and phenylalanine.

37. The composition of claim 21, wherein the weight ratio of the intra-granular polymer to the active ingredient is from about 1:1 to about 10:1.

38. The composition of claim 37, wherein the weight ratio of the intra-granular polymer to the active ingredient is from about 1:1 to 1:3.

39. The composition of claim 37, wherein the hydrophilic intra-granular polymer has a viscosity in the range of about 100 to about 5000 cps.

40. The composition of claim 39, wherein the intra-granular polymer comprises HPMC K100LV.
